(19) United States
(12) Patent Application Publication
(10) Pub. No.: US 2014/0161742 A1
(43) Pub. Date: Jun. 12, 2014

(54) ORAL COMPOSITION

(71) Applicant: SNU R&DB FOUNDATION, Seoul (KR)

(72) Inventors: Kug Sun HONG, Seoul (KR); Ki Tae NAM, Seoul (KR); Hae Lin JANG, Seoul (KR); Kyoung Suk JIN, Bucheon-si (KR); Seon Ae SHIN, Cheongwon-gun (KR); Hui Yun JEONG, Daegu (KR)

(73) Assignee: SNU R&DB FOUNDATION, Seoul (KR)

(21) Appl. No.: 13/761,053

(22) Filed: Feb. 6, 2013

(30) Foreign Application Priority Data
Dec. 11, 2012 (KR) 10-2012-0143630

(51) Int. Cl. A61K 8/24; A61Q 11/00; A61K 8/21

(52) U.S. Cl. CPC A61K 8/24; A61K 8/21; A61Q 11/00
USPC 424/52

(57) ABSTRACT

This invention relates to oral composition. The oral composition comprises whitlockite. The oral composition may further comprise fluoride ion. The whitlockite may have a chemical formula represented by Ca_{20-y}X_{y}(HPO₄)₂(PO₄)₁₂, and a ratio of Ca:X:P may be (1.28±0.2):(0.14±0.02):1. The oral composition may have good cleaning effect and tooth decay prevention effect.

### BACKGROUND

1. Technical Field

The present disclosure relates to oral composition.

2. Description of the Related Art

Generally a toothpaste includes an abrasive to remove residue, etc. from teeth physically. Calcium hydrogen phosphate, precipitated calcium carbonate, silicon dioxide (silica), insoluble sodium metaphosphate, etc. has been used as the abrasive, however, there is a problem that the enamel and dentin of teeth may be worn out if the toothpaste including the abrasive is used because the hardness of the abrasive is greater than that of apatite carbonate that is teeth enamel.

A toothpaste including hydroxyapatite (HAP: Ca₁₀(PO₄)(OH)₂) as an abrasive has been proposed to solve the above problem. The hydroxyapatite can have good cleaning effect for teeth because the hydroxyapatite can be manufactured as nano-sized particles and have a big specific surface area. However, the hydroxyapatite has hydroxyl group and the hydroxyl group reacts with fluoride ion to form apatite fluoride. The toothpaste effect may be lowered because the fluoride ion cannot have tooth decay prevention effect if the fluoride ion is contained with the hydroxyl group in the toothpaste

β-tricalcium phosphate (TCP: Ca₃(PO₄)₂) that is calcium phosphate based compound and does not include hydroxyl group has been proposed as an abrasive to solve the above problem. However, the β-tricalcium phosphate cannot have good cleaning effect for teeth because the β-tricalcium phosphate has a small specific surface area

### SUMMARY

In one embodiment, the present disclosure provides oral composition having good cleaning effect.

According to some embodiments, the present disclosure also concerns oral composition having both good cleaning effect and tooth decay prevention effect.

According to some embodiments, the present disclosure provides oral composition comprising whitlockite.

The oral composition may further comprise fluoride ion.

The fluoride ion may be contained in the oral composition in the form of fluoride compound including one or more chosen from sodium phosphate fluoride, sodium fluoride, amine fluoride, and tin fluoride.

In the oral composition, the whitlockite may be contained in an amount of 1˜40 wt % and the fluoride compound may be contained in an amount of 0.01˜1 wt % based on the total weight of the oral composition.

The whitlockite may have a chemical formula represented by Ca_{20-y}X_{y}(HPO₄)₂(PO₄)₁₂, and a ratio of Ca:X:P may be (1.28±0.2):(0.14±0.02):1.

The whitlockite powder may have a particle size of 100 nm or less.

The whitlockite may be whitlockite nanoparticles manufactured by a manufacturing method comprising adding, to water, a calcium ion supplying material and a cation supplying material containing a cation (X) other than a calcium ion to prepare a cation aqueous solution, adding a phosphoric acid supplying material to the cation aqueous solution, and aging the cation aqueous solution including the phosphoric acid supplying material.

In the cation aqueous solution, the cation (X) may be contained in an amount of 10˜50 mol % based on the total amount of cations (Ca+X).

The phosphoric acid supplying material may be added to bring a molar ratio of anion to cation (anion/cation=P/(Ca+X)) to 0.6 or greater.

The amount of the cation (X) and the molar ratio of anion to cation may be selected within a range that suppresses formation of a byproduct other than the whitlockite in view of the correlation therebetween.

The calcium ion supplying material may include one or more chosen from calcium hydroxide, calcium acetate, calcium carbonate, and calcium nitrate.

The cation (X) may include one or more chosen from Mg, Co, Sb, Fe, Mn, Y, Eu, Cd, Nd, Na, La, Sr, Pb, Ba and K.

The cation supplying material may include one or more chosen from a hydroxide compound (X-hydroxide), an acetate compound (X-acetate), a carbonate compound α-carbonate), and a nitrate compound (X-nitrate).

The phosphoric acid supplying material may include one or more chosen from diammonium hydrogen phosphate, ammonium phosphate, and phosphoric acid.

The phosphoric acid supplying material may be added in a dropwise manner.

The pH of the cation aqueous solution may be gradually decreased depending on the addition of the phosphoric acid supplying material, and the cation aqueous solution including the phosphoric acid supplying material added thereto may be aged in an acidic environment.

The manufacturing method may further comprise adding an oxidant to the water or the cation aqueous solution before adding the phosphoric acid supplying material. The oxidant may be hydrogen peroxide.

The cation supplying material may be magnesium hydroxide, the amount of magnesium (Mg) in the cation aqueous solution may be 10˜35 mol % based on the total amount of cations (Ca+Mg), and the phosphoric acid supplying material may be added to bring the molar ratio of anion to cation (anion/cation=P/(Ca+Mg)) to 0.8 or greater.

The cation supplying material may be magnesium nitrate, and the phosphoric acid supplying material may be added to bring the molar ratio of anion to cation (anion/cation=P/(Ca+Mg)) to 0.6 or greater.

The manufacturing method may further comprise drying the aged aqueous solution to form the whitlockite nanoparticles.

### BRIEF DESCRIPTION OF THE DRAWINGS

The above and other objects, features and advantages of the present invention will be more clearly understood from the following detailed description taken in conjunction with the accompanying drawings, in which:

FIG. 1 schematically illustrates a process for manufacturing whitlockite according to an embodiment of the present invention;

FIG. 2 illustrates the correlation between the cation content and the molar ratio of anion to cation in examples of the invention;

FIG. 3 illustrates an X-ray diffraction (XRD) graph of powder formed for differing amounts of cation under the condition of the molar ratio of anion to cation being fixed to 1;

FIG. 4 illustrates field emission scanning electron microscope (FESEM) images of whitlockite powder manufactured in examples of the invention;

FIG. 5 illustrates an FESEM image and a transmission electron microscope (TEM) image of whitlockite powder manufactured in Example 1 of the invention;

FIG. 6 illustrates a high resolution transmission electron microscope (HRTEM) image of the whitlockite powder manufactured in Example 1 of the invention;

FIG. 7 illustrates a graph showing a distance between planes corresponding to the lattice spacing of the whitlockite powder of FIG. 6;

FIG. 8 illustrates an XRD graph of the whitlockite powder manufactured in Example 1 of the invention and calcium magnesium phosphate powder manufactured using a solid state process;

FIG. 9 illustrates a thermal gravimetric analysis (TGA) graph of the whitlockite powder manufactured in Example 1 of the invention and the calcium magnesium phosphate powder manufactured using a solid state process; and

FIG. 10 illustrates a Fourier transform infrared (FT-IR) graph of the whitlockite powder manufactured in Example 1 of the invention and the calcium magnesium phosphate powder manufactured using a solid state process.

FIG. 11 illustrates fluoride ion content in the toothpastes manufactured in Example 5, Comparison example 1 and Comparison example 2.

### DESCRIPTION OF SPECIFIC EMBODIMENTS

Hereinafter, a detailed description will be given of embodiments of the present invention. The present invention is not limited to these embodiments and may be embodied in the other forms. The embodiments of the present invention are provided so that thorough and complete contents are ensured and the spirit of the invention is sufficiently transferred to a person having ordinary knowledge in the art.

Oral composition according to embodiments of the present invention may comprise whitlockite. The whitlockite may have a chemical formula represented by Ca_{20-y}X_{y}(HPO₄)₂(PO₄)₁₂. In this chemical formula, X may include one or more chosen from Mg, Co, Sb, Fe, Mn, Y, Eu, Cd, Nd, Na, La, Sr, Pb, Ba and K. X may have an ionic radius similar to that of the calcium ion. The whitlockite may be contained in an amount of 1˜40 wt % based on the total weight of the oral composition.

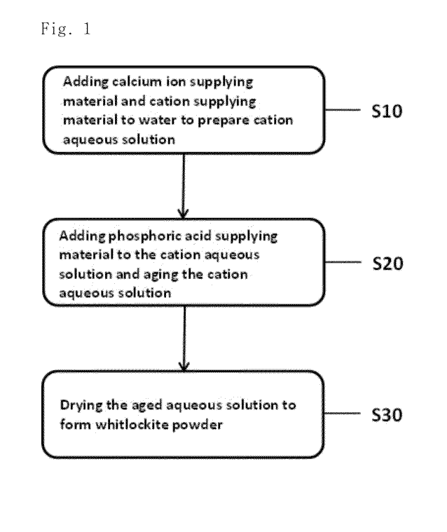

FIG. 1 schematically illustrates a process for manufacturing whitlockite according to an embodiment of the present invention.

As illustrated in FIG. 1, the method for manufacturing whitlockite may include adding a calcium ion supplying material and a cation supplying material to water to prepare a cation aqueous solution at step S10, adding a phosphoric acid supplying material to the cation aqueous solution, aging the cation aqueous solution including the phosphoric acid supplying material at step S20, and drying the aged aqueous solution to form whitlockite powder at step S30.

Specifically, the calcium ion supplying material and the cation supplying material containing a cation (X) other than a calcium ion are added to water to prepare the cation aqueous solution at step S10.

As such, the temperature of water may be equal to or lower than the boiling point, for example, 20˜100° C.

The calcium ion supplying material may include one or more chosen from calcium hydroxide, calcium acetate, calcium carbonate, and calcium nitrate.

The cation (X) may have an ionic radius similar to that of the calcium ion. The cation (X) may include one or more chosen from Mg, Co, Sb, Fe, Mn, Y, Eu, Cd, Nd, Na, La, Sr, Pb, Ba and K. The cation supplying material may include one or more chosen from a hydroxide compound (X-hydroxide), an acetate compound (X-acetate), a carbonate compound (X-carbonate), and a nitrate compound (X-nitrate).

The amount of the cation (X) in the cation aqueous solution may be 10˜50 mol % based on the total amount of cations (Ca+ X). The amount of the cation (X) contained in the final product, that is, whitlockite, is about 10 mol % based on the total amount of the calcium ion and the cation (X) contained in whitlockite. If the amount of the cation (X) in the cation aqueous solution is less than 10 mol % or is greater than 50 mol %, it is difficult to obtain very pure whitlockite.

In the case where the cation supplying material is magnesium hydroxide, the amount of magnesium (Mg) in the cation aqueous solution may be 10˜35 mol % based on the total amount of cations (Ca+Mg).

The phosphoric acid supplying material is added to the cation aqueous solution, and the cation aqueous solution including the phosphoric acid supplying material is aged at step S20.

The phosphoric acid supplying material may include one or more chosen from diammonium hydrogen phosphate, ammonium phosphate, and phosphoric acid.

The phosphoric acid supplying material may be added in a dropwise manner. When the phosphoric acid supplying material is added in a dropwise manner in this way, the pH of the cation aqueous solution may be gradually decreased. The cation aqueous solution is basic before the addition of the phosphoric acid supplying material, and then the pH thereof is lowered due to the addition of the phosphoric acid supplying material and thus a final acidic environment is formed and then the cation aqueous solution may be aged. Thereby, very pure whitlockite may be obtained. In the case where an acidic environment is provided from the beginning and thus aging progresses, calcium phosphate based compounds such as dicalcium phosphate anhydride (DCPA, CaHPO₄) and dicalcium phosphate dehydrate (DCPD, CaHPO₄.2H₂O) may be rapidly produced and may therefore remain behind. Also in the case when aging is carried out in a neutral or basic environment, the HAP phase is preferentially formed, making it difficult to synthesize very pure whitlockite. However, in the present invention, as the phosphoric acid supplying material is added in a dropwise manner to the cation aqueous solution, the synthesis proceeds in a basic environment in the early stage, and thus DCPA and DCPD phases are not formed and only the HAP phase is produced. After completion of the addition of the phosphoric acid supplying material, synthesis proceeds in an acidic environment and a whitlockite phase is formed. Also, the HAP phase formed in the basic environment is dissolved in the acidic environment and thus converted into a whitlockite phase, thereby obtaining very pure whitlockite.

The phosphoric acid supplying material may be added to bring the molar ratio of anion to cation (anion/cation=P/(Ca+X)) to 0.6 or greater. If the molar ratio of anion to cation is less than 0.6, it is difficult to make an acidic environment after addition of the phosphoric acid supplying material to the cation aqueous solution. The molar ratio of anion to cation may be appropriately set within the range that may form an acidic environment of the cation aqueous solution to which the phosphoric acid supplying material was added. Also, the molar ratio of anion to cation may be properly selected depending on the kind of phosphoric acid supplying material. For example, in the case where the cation supplying material is magnesium hydroxide, the phosphoric acid supplying material may be added to bring the molar ratio of anion to cation (anion/cation=P/(Ca+Mg)) to 0.8 or greater. In the case where the cation supplying material is magnesium nitrate, the phosphoric acid supplying material may be added to bring the molar ratio of anion to cation (anion/cation=P/(Ca+Mg)) to 0.6 or greater.

The aging time may be determined in consideration of the aging temperature and the particle size of the resulting whitlockite. For example, when the aging temperature is 80° C. and 70° C., the aging time may be 6 hr and 12 hr, respectively.

The method for manufacturing whitlockite may further include adding an oxidant to the cation aqueous solution before adding the phosphoric acid supplying material. The oxidant may be hydrogen peroxide. The addition of hydrogen peroxide may shorten the aging time. For example, when hydrogen peroxide is added, the aging time may be shortened to 30 min at an aging temperature of 80° C.

The aged aqueous solution is dried thus obtaining whitlockite powder at step S30. The whitlockite powder may be formed by subjecting the aged aqueous solution to filter pressing and then lyophilization (freeze-drying).

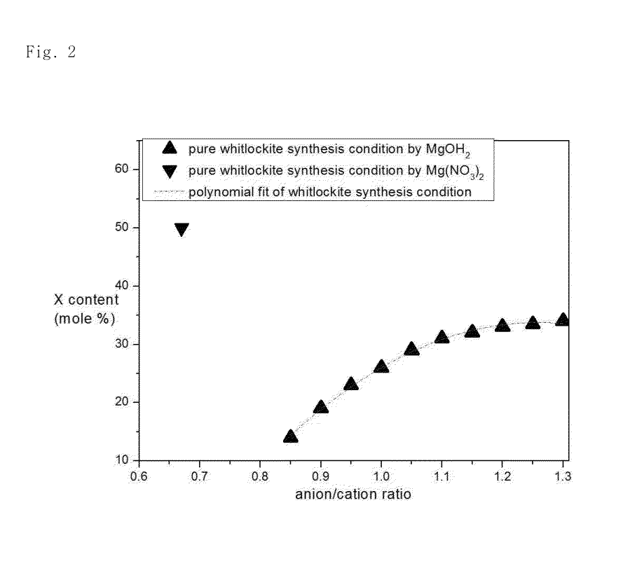

FIG. 2 illustrates the correlation between the cation content and the molar ratio of anion to cation in examples of the invention. As illustrated in FIG. 2, the horizontal axis indicates the molar ratio of anion to cation (anion/cation=P/(Ca+X)), and the vertical axis indicates the amount (mol %) of cation (X) based on the total amount of cations (Ca+X). In FIG. 2, in the case when the calcium ion supplying material is calcium hydroxide (Ca(OH)₂), the cation (X) supplying material is magnesium hydroxide (Mg(OH₂) and the phosphoric acid supplying material is phosphoric acid (H₃PO₄) and in the case when the calcium ion supplying material is calcium nitrate (Ca(NO₃)₂), the cation (X) supplying material is magnesium nitrate (Mg(NO₃)₂) and the phosphoric acid supplying material is phosphoric acid (H₃PO₄), the correlation between the amount of the magnesium ion and the molar ratio of anion to cation (P/(Ca+Mg)) is depicted.

With reference to FIG. 2, the mark ▴ designates the position when very pure whitlockite is synthesized without forming byproducts other than whitlockite, for example, secondary phases such as DCPA, HAP and so on when the cation (X) supplying material is magnesium hydroxide (Mg(OH)₂). There is a predetermined correlation between the amount of magnesium ion (X) and the molar ratio of anion to cation for forming very pure whitlockite. For example, in the case where the amount of the magnesium ion is 23 mol % and the molar ratio of anion to cation is 0.95 and in the case where the amount of the magnesium ion is 31 mol % and the molar ratio of anion to cation is 1.1, byproducts are not formed and very pure whitlockite may be obtained. However, if the magnesium ion is excessively present at a position far away from ▴, a secondary phase such as a magnesium phosphate compound may be formed. In contrast, if the magnesium ion is deficient and thus the calcium ion is comparatively excessively present, a secondary phase such as DCPA or the like may be formed, and the amount of the resulting whitlockite may decrease.

The mark ▾ designates the position where very pure whitlockite is synthesized without forming byproducts other than whitlockite when the cation (X) supplying material is magnesium nitrate (Mg(NO₃)₂). That is, there is a predetermined correlation between the amount of the magnesium ion (X) and the molar ratio of anion to cation for forming very pure whitlockite. For example, in the case where the amount of the magnesium ion is 50 mol % and the molar ratio of anion to cation is 0.67, byproducts are not formed and very pure whitlockite may result.

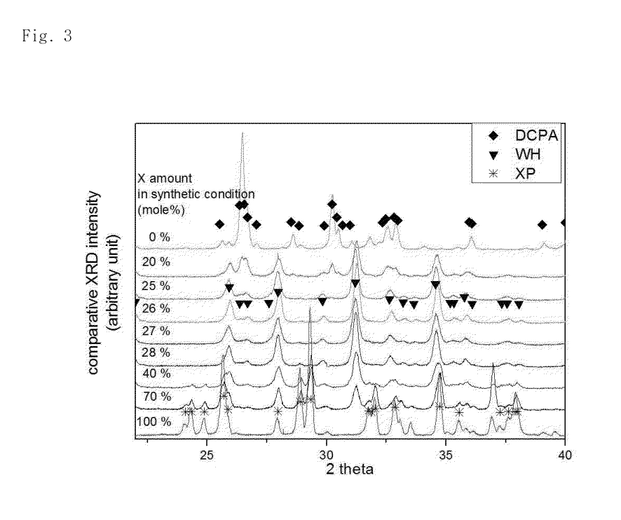

FIG. 3 illustrates an XRD graph of the powder formed for differing amounts of cation under the condition of the molar ratio of anion to cation being fixed to 1. Such powder was manufactured using calcium hydroxide, magnesium hydroxide and phosphoric acid as the calcium ion supplying material, the cation (X) supplying material and the phosphoric acid supplying material, respectively.

With reference to FIG. 3, in the case where the molar ratio of anion to cation is 1, when the amount of the magnesium ion as the cation (X) is 26 mol % and the amount of the calcium ion is 74 mol %, the purest whitlockite (WH) is formed. If the amount of the magnesium ion is less than 26 mol %, DCPA is formed as a secondary phase. If the magnesium ion is not contained and the amount of the calcium ion is 100 mol %, pure DCPA is formed. In contrast, if the amount of the magnesium ion is greater than 26 mol %, magnesium phosphate (XP) is formed as a secondary phase, and if the amount of the magnesium ion is 100 mol %, pure magnesium phosphate is formed.

A better understanding of the present invention may be obtained via the following examples, which are set forth to illustrate, but are not to be construed as limiting the present invention.

### EXAMPLE

### Example 1

With reference to the correlation between the cation content and the molar ratio of anion to cation as illustrated in FIG. 2, the amount of the magnesium ion was set to 23 mol % based on the total amount of cations and the molar ratio of anion to cation (P/(Ca+Mg)) was set to 0.95, and aging was performed at 80° C., thus synthesizing whitlockite.

Tertiary distilled water was boiled to remove dissolved gaseous impurities, after which 0.385 mol calcium hydroxide (0.5 mol multiplied by 0.77) and 0.115 mol magnesium hydroxide (0.5 mol multiplied by 0.23) were added to the distilled water, and stirring was then performed at 80° C., thus preparing a calcium-magnesium aqueous solution.

0.475 mol phosphoric acid (0.5 mol multiplied by 0.95) was placed in a burette and then slowly added in a dropwise manner to the calcium-magnesium aqueous solution which was being stirred. After the completion of the addition of phosphoric acid to the calcium-magnesium aqueous solution, the solution was aged while being stirred at 80° C. for 6 hr, thereby synthesizing whitlockite.

The aged aqueous solution was filter pressed and lyophilized, yielding whitlockite powder.

### Example 2

With reference to the correlation between the cation content and the molar ratio of anion to cation as illustrated in FIG. 2, the amount of the magnesium ion was set to 31 mol % based on the total amount of cations and the molar ratio of anion to cation (P/(Ca+Mg)) was set to 1.1, and aging was performed at 70° C., thus synthesizing whitlockite.

Tertiary distilled water was boiled to remove dissolved gaseous impurities, after which 0.345 mol calcium hydroxide (0.5 mol multiplied by 0.69) and 0.155 mol magnesium hydroxide (0.5 mol multiplied by 0.31) were added to the distilled water, and stirring was then performed at 70° C., thus preparing a calcium-magnesium aqueous solution.

0.55 mol phosphoric acid (0.5 mol multiplied by 1.1) was placed in a burette and then slowly added in a dropwise manner to the calcium-magnesium aqueous solution which was being stirred. After the completion of the addition of phosphoric acid to the calcium-magnesium aqueous solution, the solution was aged while being stirred at 70° C. for 12 hr, thereby synthesizing whitlockite.

The aged aqueous solution was filter pressed and lyophilized, yielding whitlockite powder.

### Example 3

With reference to the correlation between the cation content and the molar ratio of anion to cation as shown in FIG. 2, the amount of the magnesium ion was set to 23 mol % based on the total amount of cations and the molar ratio of anion to cation (P/(Ca+Mg)) was set to 0.95, and aging was performed in a hydrogen peroxide aqueous solution at 80° C., thus synthesizing whitlockite.

Tertiary distilled water was boiled to remove dissolved gaseous impurities, after which a 10% hydrogen peroxide aqueous solution was added in an amount of 30 wt % based on the total weight to the distilled water. As such, the concentration and the amount of the added hydrogen peroxide aqueous solution may be set so as to accelerate the synthesis of whitlockite to thereby shorten the aging time, and these concentration and amount are not limited to those of Example 3. 0.385 mol calcium hydroxide (0.5 mol multiplied by 0.77) and 0.115 mol magnesium hydroxide (0.5 mol multiplied by 0.23) were added to the distilled water which the hydrogen peroxide aqueous solution was added to, and then stirring was performed at 80° C., thus preparing a calcium-magnesium aqueous solution.

0.475 mol phosphoric acid (0.5 mol multiplied by 0.95) was placed in a burette and then slowly added in a dropwise manner to the calcium-magnesium aqueous solution which was being stirred. After the completion of the addition of phosphoric acid to the calcium-magnesium aqueous solution, the solution was aged while being stirred at 80° C. for 30 min, thereby synthesizing whitlockite.

The aged aqueous solution was filter pressed and lyophilized, yielding whitlockite powder.

### Example 4

With reference to the correlation between the cation content and the molar ratio of anion to cation as shown in FIG. 2, the amount of the magnesium ion was set to 50 mol % based on the total amount of cations and the molar ratio of anion to cation (P/(Ca+Mg)) was set to 0.67, and aging was performed at 80° C., thus synthesizing whitlockite.

Tertiary distilled water was boiled to remove dissolved gaseous impurities, after which 0.25 mol calcium nitrate (Ca(NO₃)₂) (0.5 mol multiplied by 0.5) and 0.25 mol magnesium nitrate (Mg(NO₃)₂) (0.5 mol multiplied by 0.5) were added to the distilled water, and then stirring was performed at 80° C., thus preparing a calcium-magnesium aqueous solution.

0.335 mol phosphoric acid (0.5 mol multiplied by 0.67) was placed in a burette and then slowly added in a dropwise manner to the calcium-magnesium aqueous solution which was being stirred. After the completion of the addition of phosphoric acid to the calcium-magnesium aqueous solution, the solution was aged while being stirred at 80° C. for 9 hr, thereby synthesizing whitlockite.

The aged aqueous solution was filter pressed and lyophilized, yielding whitlockite powder.

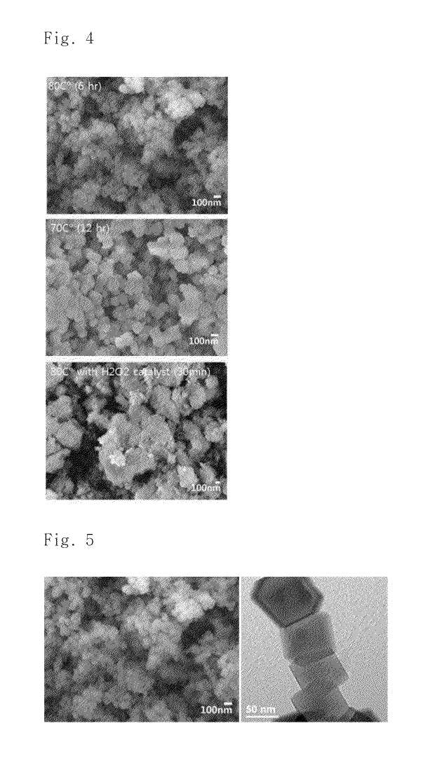

FIG. 4 illustrates FESEM images of whitlockite powder manufactured in the examples of the invention.

With reference to FIG. 4, four images respectively show FESEM images of whitlockite powder of Examples 1 to 3. The particle size of whitlockite powder of Examples 1 to 3 is smaller than 100 nm. The particle size of the whitlockite powder may be controlled depending on the aging conditions. Furthermore, the whitlockite powder may be obtained via aging at 80° C. for 6 hr. As such, when hydrogen peroxide is added, the aging time may be shortened to 30 min.

FIG. 5 illustrates an FESEM image and a TEM image of the whitlockite powder of Example 1 of the invention.

With reference to FIG. 5, the left image shows an FESEM image, and the right image shows a TEM image. The whitlockite powder of Example 1 has a rhombohedral shape having a uniform size of about 50 nm.

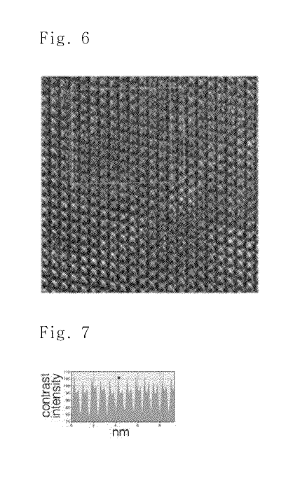

FIG. 6 is an HRTEM image of the whitlockite powder of Example 1 of the invention, and FIG. 7 is a graph showing the distance between planes corresponding to the lattice spacing of the whitlockite powder of FIG. 6.

With reference to FIGS. 6 and 7, the whitlockite powder of Example 1 can be seen to regularly have only an intrinsic distance (8.067 Å-whitlockite (012)) between planes of whitlockite as represented by JCPDS (70-2064). That is, the whitlockite powder of Example 1 can be seen to have high purity.

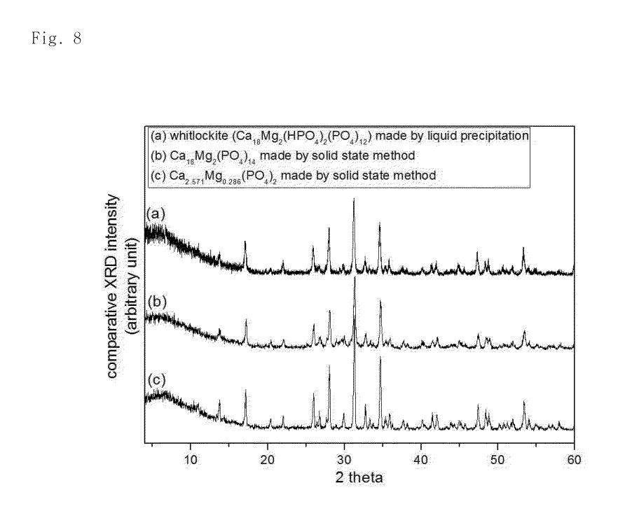

FIG. 8 is an XRD graph of the whitlockite powder of Example 1 of the invention and the calcium magnesium phosphate powder obtained using a solid state process. The graph (a) is an XRD graph of the whitlockite powder of Example 1, and the graph (b) is an XRD graph of calcium magnesium phosphate powder obtained via heat treatment at 1100° C. using a solid state process in which the ratio of Ca:Mg:P is the same as that of the whitlockite powder of Example 1. The graph (c) is an XRD graph of calcium magnesium phosphate powder obtained via heat treatment at 1100° C. using a solid state process in which the ratio of (Ca+Mg):P is 3:2 (which is the same as the ratio of Ca:P of TCP) and which has the same ratio of Ca:Mg as that of the whitlockite powder of Example 1.

With reference to FIG. 8, the whitlockite powder of Example 1 manufactured using liquid precipitation shows the XRD peak in the same form as in the powder synthesized using a solid state process. Because the powder synthesized using a solid state process shows only the peaks of rhombohedral crystals of whitlockite and TCP on XRD without a secondary phase, the whitlockite powder of Example 1 can be confirmed to be pure whitlockite powder having no secondary phase.

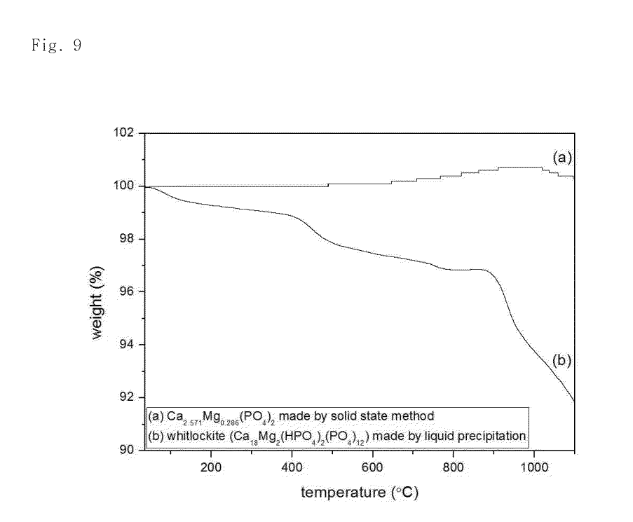

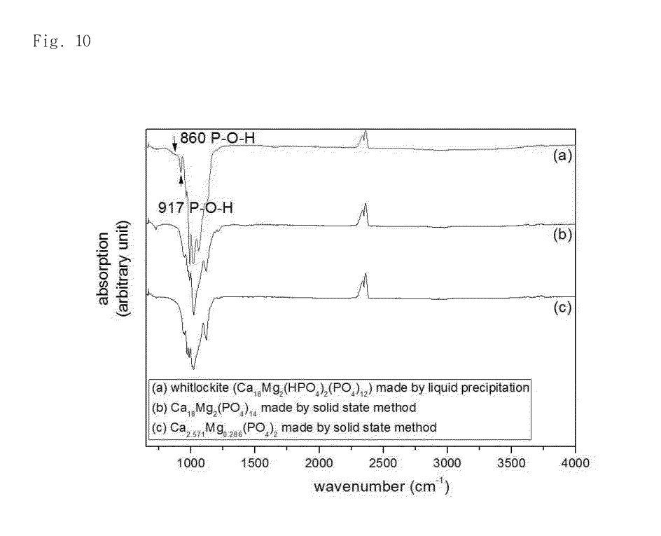

FIG. 9 is a TGA graph of the whitlockite powder of Example 1 of the invention and the calcium magnesium phosphate powder obtained using a solid state process. The graph (a) shows the weight of powder measured at a heating rate of 10° C./min, the powder being calcium magnesium phosphate powder obtained via heat treatment at 1100° C. using a solid state process in which the ratio of (Ca+Mg):P is 3:2 (which is the same as the ratio of Ca:P of TCP) and which has the same ratio of Ca:Mg as that of the whitlockite powder of Example 1. The graph (b) shows the weight of the whitlockite powder of Example 1 measured at a heating rate of 10° C./min.

With reference to FIG. 9, as the temperature is increased, the weight of the calcium magnesium phosphate powder manufactured using a solid state process is almost uniform, whereas the weight of the whitlockite powder of Example 1 is remarkably decreased. Because the whitlockite powder of Example 1 contains hydrogen, its weight is decreased due to dehydration in proportion to the increase in temperature, but the calcium magnesium phosphate powder manufactured using a solid state process has no hydrogen and thus its weight does not change even when the temperature is increased.

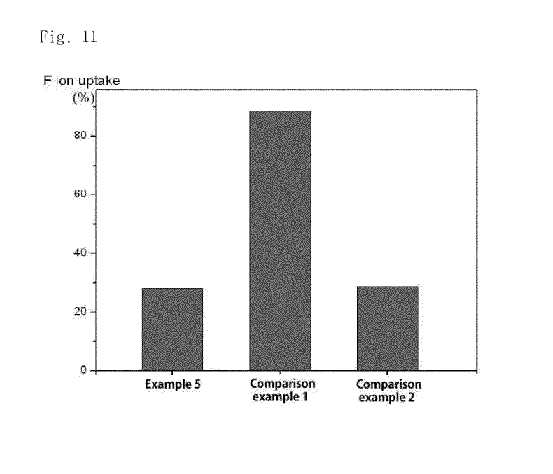

FIG. 10 is an FT-IR graph of the whitlockite powder of Example 1 of the invention and the calcium magnesium phosphate powder manufactured using a solid state process. In FIG. 10, the horizontal axis indicates a wave number and the vertical axis indicates a relative absorbance. The graph (a) is an FT-IR graph of the whitlockite powder of Example 1 and the graph (b) is an FT-IR graph of the calcium magnesium phosphate powder obtained via heat treatment at 1100° C. using a solid state process in which the ratio of Ca:Mg:P is the same as that of the whitlockite powder of Example 1. The graph (c) is an FT-IR graph of the calcium magnesium phosphate powder obtained via heat treatment at 1100° C. using a solid state process in which the ratio of (Ca+Mg):P is 3:2 (which is the same as the ratio of Ca:P of TCP) and which has the same ratio of Ca:Mg as that of the whitlockite powder of Example 1.

With reference to FIG. 10, the composition and bonding structure of the whitlockite powder of Example 1 are comparatively similar to those of the calcium magnesium phosphate powder manufactured using a solid state process, but this whitlockite powder may have an HPO₄ bonding structure, unlike the calcium magnesium phosphate powder manufactured via heat treatment at high temperature using a solid state process. As shown in FIG. 10, the whitlockite powder of Example 1 can be seen to have a P—O—H bond.

When analyzing whitlockite of the examples of the invention with inductively coupled plasma (ICP), a ratio of Ca:X:P is shown to be (1.28±0.2):(0.14±0.02):1, which is very similar to 1.28:0.14:1 which is the theoretical value of whitlockite. In the chemical formula, Ca_{20-y}X_{y}(HPO₄)₂(PO₄)₁₂, the ratio of Ca:X:P, (20-y):y:14 may be (1.28±0.2):(0.14±0.02):1.

According to embodiments of the present invention, the whitlockite can be simply manufactured without performing heat treatment at high temperature and washing to remove additional ions. The manufacturing process can be simplified and thus the manufacturing cost can be reduced. Also, nano-sized whitlockite powder having high purity, high crystallinity, and a particle size of 100 nm or less can be mass produced. The oral composition comprising the whitlockite as an abrasive has good cleaning effect, especially for teeth, because the whitlockite has big specific surface area as nano-sized powder.

The oral composition may further comprise fluoride ion. The fluoride ion may be added to the oral composition in the form of fluoride compound. The fluoride compound may include one or more chosen from sodium phosphate fluoride, sodium fluoride, amine fluoride, and tin fluoride. The fluoride compound may be contained in an amount of 0.01˜1 wt % based on the total weight of the oral composition.

The whitlockite and the fluoride ion do not react with each other and can coexist in the oral composition because the whitlockite does not include hydroxyl group. The oral composition can have tooth decay prevention effect by including the fluoride ion

The oral composition may further comprise an abrasive other than the whitlockite, a solvent, a bonding agent, a diluting agent, a preservative, a detergent, an oral treatment agent, and/or a flavoring agent. The abrasive may include dental type silica, apatite carbonate, etc. The solvent may include concentrated glycerin, purified water, etc. The bonding agent may include carboxymethylcellulose, carrageenan, etc. The diluting agent may include light anhydrous silicic acid, etc. The preservative may include methyl paraoxy benzoate, etc. The detergent may include sodium lauryl sulfate. The oral treatment agent may include a gingivitis treatment agent, etc. such as tocopherol acetate. The flavoring agent may include herb mint, natural eucalyptus, etc.

The oral composition may be manufactured in the various forms, however, toothpaste including nano-sized whitlockite having a particle size of 100 nm or less is described in the following examples

In Table 1 below, Example 5 indicates toothpaste including the nano-sized whitlockite as an abrasive, Comparison example 1 indicates toothpaste including hydroxyapatite as an abrasive, and Comparison example 2 toothpaste including tricalcium phosphate manufactured by a solid state process as an abrasive.

TABLE 1

| | | Exam- | Comparison | Comparison |
|---|---|---|---|---|
| | | ple 5 | example 1 | example 2 |
| Type | Ingredient | (wt %) | (wt %) | (wt %) |
| Abrasive | Nano-sized | 20 | — | — |
| | whitlockite | | | |
| | Hydroxyapatite | — | 20 | — |
| | Tricalcium | — | — | 20 |
| | phosphate | | | |
| | Dental type | 5 | 5 | 5 |
| | silica | | | |
| Tooth decay | Sodium phosphate | 0.5 | 0.5 | 0.5 |
| prevention | fluoride | | | |
| agent | | | | |
| Solvent | Concentrated | 46 | 46 | 46 |
| | glycerin | | | |
| | Purified water | 23 | 23 | 23 |
| Bonding | Carboxymethyl- | 1 | 1 | 1 |
| agent | cellulose | | | |
| | Carrageenan | 0.2 | 0.2 | 0.2 |
| Diluting | Light anhydrous | 1.3 | 1.3 | 1.3 |
| agent | silicic acid | | | |
| Preservative | Methyl paraoxy | 0.2 | 0.2 | 0.2 |
| | benzoate | | | |
| Detergent | Sodium lauryl | 0.5 | 0.5 | 0.5 |
| | sulfate | | | |
| Gingivitis | Tocopherol | 0.3 | 0.3 | 0.3 |
| treatment | acetate | | | |
| agent | | | | |
| Flavoring | Herb mint | 1 | 1 | 1 |
| agent | Natural | 1 | 1 | 1 |
| | eucalyptus | | | |

FIG. 11 illustrates fluoride ion content in the toothpastes manufactured in Example 5, Comparison example 1 and Comparison example 2. FIG. 11 indicates the uptake of fluoride ion in toothpaste measured 24 hours after the toothpaste had been manufactured.

As illustrated in FIG. 11, in the case of Comparison example 1, about 90% of the fluoride ion reacts with hydroxyl group of the hydroxyapatite and disappears, and about 10% of the fluoride ion remains in the toothpaste. In the cases of Example 5 and Comparison example 2, about 30% of the fluoride ion disappears and about 70% of the fluoride ion remains in the toothpaste because the whitlockite and the tricalcium phosphate do not include hydroxyl group. That is, the toothpaste including hydroxyapatite as an abrasive cannot have tooth decay prevention effect although fluoride ion is added to the toothpaste. However, the toothpaste of Example 5 of the present invention including nano-sized whitlockite can have tooth decay prevention effect because the amount of fluoride ion which remains in the toothpaste is much more than that of fluoride which disappears by uptake in the toothpaste.

Table 2 below indicates BET (Brunauer-Emmett-Teller) analysis results on the nano-sized whitlockite of Example 5, hydroxyapatite of Comparison example 1, and tricalcium phosphate of Comparison example 2.

TABLE 2

| | | | Tricalcium |
|---|---|---|---|
| | Whitiockite | Hydroxyapatite | phosphate |
| Specific surface area (m²/g) | 29 | 84 | 0.3 |

As shown in Table 2, The specific surface area of the nano-sized whitlockite is 29 m²/g. The specific surface area of the hydroxyapatite is 84 m²/g. The specific surface area of the tricalcium phosphate is 0.3 m²/g. The specific area of the nano-sized whitlockite of examples of the present invention is about 100 times larger than that of the tricalcium phosphate. The toothpaste of Example 5 can have good cleaning effect, especially for teeth because the toothpaste includes the whitlockite having the big specific area as an abrasive.

Table 3 below indicates toothpaste including nano-sized whitlockite and dental type silica

TABLE 3

| | | Exam- | Exam- | Exam- | Exam- |
|---|---|---|---|---|---|
| | | ple 6 | ple 7 | ple 8 | ple 9 |
| Type | Ingredient | (wt %) | (wt %) | (wt %) | (wt %) |
| Abrasive | Nano-sized | 5 | 15 | 25 | 35 |
| | whitlockite | | | | |
| | Dental type | 5 | 5 | 5 | 5 |
| | silica | | | | |
| Tooth decay | Sodium | 0.5 | 0.5 | 0.5 | 0.5 |
| prevention | phosphate | | | | |
| agent | fluoride | | | | |
| Solvent | Concentrated | 56 | 51 | 41 | 36 |
| | glycerin | | | | |
| | Purified water | 28 | 23 | 23 | 18 |
| Bonding | Carboxymethyl- | 1 | 1 | 1 | 1 |
| agent | cellulose | | | | |
| | Carrageenan | 0.2 | 0.2 | 0.2 | 0.2 |
| Diluting | Light anhydrous | 1.3 | 1.3 | 1.3 | 1.3 |
| agent | silicic acid | | | | |
| Preservative | Methyl paraoxy | 0.2 | 0.2 | 0.2 | 0.2 |
| | benzoate | | | | |
| Detergent | Sodium lauryl | 0.5 | 0.5 | 0.5 | 0.5 |
| | sulfate | | | | |
| Gingivitis | Tocopherol | 0.3 | 0.3 | 0.3 | 0.3 |
| treatment | acetate | | | | |
| agent | | | | | |
| Flavoring | Herb mint | 1 | 1 | 1 | 1 |
| agent | Natural | 1 | 1 | 1 | 1 |
| | eucalyptus | | | | |

As shown in Table 3, the toothpastes of Examples 6 to 9 include the nano-sized whitlockite in the amount of 5 wt %, 15 wt %, 25 wt %, and 35 wt % based on the total weight of the toothpaste. The toothpastes of Examples 6 to 9 can have better cleaning effect than the toothpastes including the tricalcium phosphate as an abrasive by the nano-sized whitlockite having the big specific surface area. Also, the toothpastes of Examples 6 to 9 can have better tooth decay prevention effect than the toothpastes including the hydroxyapatite and the fluoride compound.

TABLE 4

| | | Example 10 | Example 11 | Example 12 | Example 13 | Example 14 |
|---|---|---|---|---|---|---|
| Type | Ingredient | (wt %) | (wt %) | (wt %) | (wt %) | (wt %) |
| Abrasive | Nano-sized whitlockite | 1 | 10 | 20 | 30 | 40 |
| | Apatite carbonate | 39 | 30 | 20 | 10 | — |
| | Dental type silica | 5 | 5 | 5 | 5 | 5 |
| Tooth decay | Sodium phosphate fluoride | 0.5 | 0.5 | 0.5 | 0.5 | 0.5 |
| prevention agent | | | | | | |
| Solvent | Concentrated glycerin | 36 | 36 | 36 | 36 | 36 |
| | Purified water | 13 | 13 | 13 | 13 | 13 |
| Bonding agent | Carboxymethylcellulose | 1 | 1 | 1 | 1 | 1 |
| | Carrageenan | 0.2 | 0.2 | 0.2 | 0.2 | 0.2 |
| Diluting agent | Light anhydrous silicic | 1.3 | 1.3 | 1.3 | 1.3 | 1.3 |
| | acid | | | | | |
| Preservative | Methyl paraoxy benzoate | 0.2 | 0.2 | 0.2 | 0.2 | 0.2 |
| Detergent | Sodium lauryl sulfate | 0.5 | 0.5 | 0.5 | 0.5 | 0.5 |
| Gingivitis | Tocopherol acetate | 0.3 | 0.3 | 0.3 | 0.3 | 0.3 |
| treatment agent | | | | | | |
| Flavoring | Herb mint | 1 | 1 | 1 | 1 | 1 |
| agent | Natural eucalyptus | 1 | 1 | 1 | 1 | 1 |

As shown in Table 4, the toothpastes of Examples 10 to 14 include the abrasives including the nano-sized whitlockite, the apatite carbonate, and the dental type silica in the amount of 45 wt % and the nano-sized whitlockite in the amount of 1 wt %, 10 wt %, 20 wt %, 30 wt %, and 40 wt % based on the total weight of the toothpaste. The toothpastes of Examples 10 to 14 can have better cleaning effect than the toothpastes including the tricalcium phosphate as an abrasive by the nano-sized whitlockite having the big specific surface area. Also, the toothpastes of Examples 10 to 14 can have better tooth decay prevention effect than the toothpastes including the hydroxyapatite and the fluoride compound.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. Oral composition comprising whitlockite.

2. The oral composition of claim 1, further comprising fluoride ion.

3. The oral composition of claim 2, wherein the fluoride ion is contained in the oral composition in the form of fluoride compound including one or more chosen from sodium phosphate fluoride, sodium fluoride, amine fluoride, and tin fluoride.

4. The oral composition of claim 3, wherein, in the oral composition, the whitlockite is contained in an amount of 1˜40 wt % and the fluoride compound is contained in an amount of 0.01˜1 wt % based on the total weight of the oral composition.

5. The oral composition of claim 1, wherein the whitlockite has a chemical formula represented by Ca_{20-y}X_{y}(HPO₄)₂(PO₄)₁₂, and a ratio of Ca:X:P is (1.28±0.2):(0.14±0.02):1.

6. The oral composition of claim 1, wherein the whitlockite powder has a particle size of 100 nm or less.

7. The oral composition of claim 1, wherein the whitlockite is whitlockite nanoparticles manufactured by a manufacturing method comprising,
adding, to water, a calcium ion supplying material and a cation supplying material containing a cation (X) other than a calcium ion to prepare a cation aqueous solution,
adding a phosphoric acid supplying material to the cation aqueous solution, and
aging the cation aqueous solution including the phosphoric acid supplying material.

8. The oral composition of claim 7, wherein, in the cation aqueous solution, the cation (X) is contained in an amount of 10˜50 mol % based on a total amount of cations (Ca+X).

9. The oral composition of claim 7, wherein the phosphoric acid supplying material is added to bring a molar ratio of anion to cation (anion/cation=P/(Ca+X)) to 0.6 or greater.

10. The oral composition of claim 9, wherein an amount of the cation (X) and the molar ratio of anion to cation are selected within a range that suppresses formation of a byproduct other than the whitlockite in view of a correlation therebetween.

11. The oral composition of claim 7, wherein the calcium ion supplying material includes one or more chosen from calcium hydroxide, calcium acetate, calcium carbonate, and calcium nitrate.

12. The oral composition of claim 7, wherein the cation (X) includes one or more chosen from Mg, Co, Sb, Fe, Mn, Y, Eu, Cd, Nd, Na, La, Sr, Pb, Ba and K.

13. The oral composition of claim 7, wherein the cation supplying material includes one or more chosen from a hydroxide compound (X-hydroxide), an acetate compound (X-acetate), a carbonate compound (X-carbonate), and a nitrate compound (X-nitrate).

14. The oral composition of claim 7, wherein the phosphoric acid supplying material includes one or more chosen from diammonium hydrogen phosphate, ammonium phosphate, and phosphoric acid.

15. The oral composition of claim 7, wherein the phosphoric acid supplying material is added in a dropwise manner.

16. The oral composition of claim 15, wherein a pH of the cation aqueous solution is gradually decreased depending on addition of the phosphoric acid supplying material, and
the cation aqueous solution including the phosphoric acid supplying material added thereto is aged in an acidic environment.

17. The oral composition of claim 7, wherein the manufacturing method further comprises adding an oxidant to the water or the cation aqueous solution before adding the phosphoric acid supplying material.

18. The oral composition of claim 7, wherein the cation supplying material is magnesium hydroxide,
an amount of magnesium (Mg) in the cation aqueous solution is 10˜35 mol % based on the total amount of cations (Ca+Mg), and
the phosphoric acid supplying material is added to bring the molar ratio of anion to cation (anion/cation=P/(Ca+Mg)) to 0.8 or greater.

19. The oral composition of claim 7, wherein the cation supplying material is magnesium nitrate, and
the phosphoric acid supplying material is added to bring the molar ratio of anion to cation (anion/cation=P/(Ca+Mg)) to 0.6 or greater.

20. The oral composition of claim 7, wherein the manufacturing method further comprises drying the aged aqueous solution to form the whitlockite nanoparticles.
